(19) United States
(12) Patent Application Publication
(10) Pub. No.: US 2016/0310515 A1
(43) Pub. Date: Oct. 27, 2016

(54) Water-Dispersible Supplements and Methods

(71) Applicant: K10 HOLDINGS LLC, New York, NY (US)

(72) Inventor: Ryan H. SINGER, New York, NY (US)

(21) Appl. No.: 14/782,977

(22) PCT Filed: Dec. 9, 2014
(86) PCT No.: PCT/US14/69304

§ 371 Date: Oct. 7, 2015

Related U.S. Application Data
(60) Provisional application No. 61/914,769, filed on Dec. 11, 2013.

(51) Int. Cl. A61K 31/7004; A61K 31/10; A61K 31/375; A61K 31/353; A61K 31/728; A61K 33/32; A61K 38/05; A61K 33/00; A61K 35/20; A61K 31/4406; A61K 36/28; A61K 31/51; A61K 31/525; A61K 31/4415; A61K 31/714; A61K 36/45; A61K 35/742; A61K 31/202; A23K 50/48; A23K 20/174; A23K 20/24; A23K 20/22; A23K 20/20; A23K 20/28; A23K 20/142; A23K 20/111; A23K 20/121; A61K 31/702

(52) U.S. Cl. CPC A61K 31/7004; A61K 31/702; A61K 31/10; A61K 31/375; A61K 31/353; A61K 31/728; A61K 33/32; A61K 38/05; A61K 33/00; A61K 35/20; A61K 31/4406; A61K 36/28; A61K 31/51; A61K 31/525; A61K 31/4415; A61K 31/714; A61K 36/45; A61K 35/742; A61K 31/202; A23K 50/48; A23K 20/174; A23K 20/24; A23K 20/22; A23K 20/30; A23K 20/28; A23K 20/142; A23K 20/111; A23K 20/121

(57) ABSTRACT

Provided is a method for delivering a supplement to a non-human subject, such as a dog. The method includes contacting a source of drinking water for the subject with an effective amount of the supplement and allowing the subject to consume the supplement containing water, wherein by consuming the water, the subject ingests a recommended daily allowance (RDA) of the supplement. Animal health, in particular dog health, supplements and kits containing such supplements are also provided. Methods of making the supplements are further provided.

### CROSS-REFERENCE TO RELATED APPLICATIONS

The present application claims priority to U.S. Provisional Patent Application No. 61/914,769, filed Dec. 11, 2013, which is incorporated herein by reference in its entirety.

### FIELD OF INVENTION

The present invention relates, inter alia, to new supplement formulations for non-human subjects, such as dogs, that may be administered to such subjects through their drinking water. More particularly, the invention concerns water-dispersible health-promoting compositions containing vitamins, minerals or other active ingredients for pets. The invention also relates to methods of preparing and using such compositions.

### BACKGROUND OF THE INVENTION

A 2006 study found that as many as a one-third (⅓) of dogs and cats in the United States may receive health-promoting agents. The most common of these agents are multivitamins, supplements to support arthritic joints, and fatty acids to reduce shedding and improve an animal coat's shine. (Freeman et al., 2006). Pet owners also may give probiotics to their pets to alleviate gastrointestinal problems or antioxidants or other joint supplements to counteract the effects of aging.

Because pets are increasingly considered members of the family, their nutritional needs are getting as much attention as other household members. Research shows that a poor diet can be as detrimental to a dog or a cat as it can be to a human. Furthermore, vitamins or nutritional supplements may also be important at different stages of an animal's life. For example, a young animal may need calcium to build strong bones and teeth or other vitamins and minerals to effectuate proper development. Similarly, older animals may need supplements to aid in digestion or to alleviate joint discomfort. In addition, older animals often absorb and store less vitamins, so a constant source of vitamins for daily requirements may be needed.

Numerous vitamin preparations for animals, including companion animals, are available on the market. These vitamin preparations contain a plurality of different vitamins and are considered to enhance and support performance and regeneration. However, these preparations suffer from several problems. Some of these preparations have a color, taste, smell and/or composition that the intended animal recipient finds unsatisfactory. In addition, these preparations are mostly in a hard tablet, soft pill, or capsule form that may make them hard to swallow. Further, the delivery of such conventional forms can be dangerous to the caregiver and stressful to the animal, if delivered manually. Delivery may fail entirely if administered in the food because the animal may not ingest the preparation as the color, smell and/or taste may be off-putting.

In view of the foregoing, there is a need for animal supplements that may be delivered to a non-human subject, such as a dog, in a safe, low-stress manner that ensures that the subject receives the proper dose of the supplement. The present invention is directed to meeting these and other needs.

### SUMMARY OF THE INVENTION

One embodiment of the present invention is a method for delivering a supplement to a non-human subject. This method comprises contacting a source of drinking water for the subject with an effective amount of the supplement and allowing the subject to consume the supplement containing water, wherein by consuming the water, the subject ingests a recommended daily allowance (RDA) of the supplement.

Another embodiment of the invention is a method for delivering, on a daily basis, a supplement to a dog. This embodiment comprises contacting a source of drinking water for the dog with an amount of the supplement sufficient to provide a recommended daily allowance (RDA) of the supplement to the dog and allowing the dog to consume the supplement-containing water, wherein by consuming the water, the dog ingests the RDA of the supplement.

Another embodiment of the invention is an animal health supplement. The animal health supplement comprises an effective amount of at least one animal health promoting active ingredient, which supplement is sufficient to deliver an effective amount of the active ingredient to the animal via the animal's drinking water, wherein upon contact with the drinking water the supplement disperses within the drinking water to a sufficient degree that the animal is able to ingest the supplement as it consumes the drinking water.

A further embodiment of the present invention is a dog health supplement. This dog health supplement comprises an effective amount of at least one dog health promoting active ingredient, which supplement is sufficient to deliver a recommended daily allowance (RDA) of the active ingredient to the dog via the dog's drinking water, wherein upon contact with the drinking water the supplement disperses within the drinking water to a sufficient degree that the dog is able to ingest the supplement as it consumes the drinking water.

The present invention also includes kits for promoting animal health, preferably dog health. These kits comprise the animal health or dog health supplements disclosed herein packaged together with instructions for use.

The present invention further includes methods of making the animal health supplement, preferably the dog health supplement, disclosed herein. These methods comprise obtaining each ingredient having a diameter between 7-750 μm and combining each ingredient for a sufficient period of time to form a mixture.

### DETAILED DESCRIPTION OF THE INVENTION

The present invention relates to novel supplements for non-human subjects, such as dogs or ferrets, and to processes for making and using such supplements. The supplements of the invention are safe for long-term consumption by non-human subjects, particularly small mammals, such as dogs and ferrets. Further, the supplements are designed to be dispersed and/or dissolved in a liquid medium, such as water. And, when dispersed and/or dissolved in such liquid medium, the supplements are substantially colorless, tasteless, and odorless, which make them an ideal vector for delivering, e.g., vitamins and minerals to a non-human subject, such as a dog or ferret, in a manner that is safe for the care-giver and stress-free for the pet. Thus, one embodiment of the present invention is a method for delivering a supplement to a non-human subject. This method comprises contacting a source of drinking water for the subject with an effective amount of the supplement and allowing the subject to consume the supplement containing water, wherein by consuming the water, the subject ingests a recommended daily allowance (RDA) of the supplement.

In the present invention, “contacting” means to bring the supplement into intimate association with a liquid medium, such as, e.g., water, so that the supplement disperses and/or dissolves, partially or completely, in the liquid medium. Contacting may be accomplished by, e.g., pouring the contents of a packet containing, e.g., a once-a-day formulation of the present invention into an subject's drinking bowl. Upon contact with the liquid medium, e.g., water, the formulation will immediately disperse and/or begin to dissolve. The process may be aided, optionally by stirring the liquid medium containing the supplement. Alternatively, the liquid medium may be placed in a vessel with a water-tight cap, the supplement added, and the cap sealed on the vessel. The vessel may then be shaken to facilitate dispersal and/or dissolution of the supplement. Thereafter, the liquid medium-containing the supplement may be poured into a subject's drinking vessel, e.g., bowl.

In the present invention, an “effective amount” of a supplement disclosed herein is an amount of such supplement that is sufficient to effect beneficial or desired results as described herein when administered to a subject. In the present invention, effective amounts may provide a RDA of a supplement to the non-human subject.

As used herein, a “recommended daily allowance” or “RDA” of a supplement may be determined by one of several organizations that publish nutrient guidelines based on the life stage of an animal, e.g. a dog. One such organization is the Association of American Feed Control Officials (AAFCO) which publishes dog nutrient profiles for adult maintenance and reproduction. The National Research Council (NRC) also publishes nutrient profiles for dogs for various life stages, most recently in 2006. Both AAFCO and NRC list minimum nutrient requirements and maximum nutrient requirements for nutrients with potential toxicity. Those of skill in the art also know the quantities of vitamins and minerals above the guidelines for a species of animal that would be harmful to the animal consuming the composition. Moreover, the present invention includes ±10% of the RDA to account for common variables including packaging variance, loss of activity during storage, etc.

In the present invention, the supplements are supplied to the drinking water of a subject. While water is a preferred source in which to disperse and/or dissolve the supplement, any substantially aqueous medium, such as a balanced salt solution or an electrolyte solution that is safe and available to a subject may be used.

In one aspect of this embodiment, the subject is selected from the group consisting of lab animals, farm animals, and domesticated animals. Preferably, the domesticated animal is selected from the group consisting of dogs, cats, rabbits, guinea pigs, hamsters, ferrets, and horses. More preferably, the domesticated animal is a dog.

In another aspect of this embodiment, the supplement is an animal health promoting product. Preferably, the animal health promoting product is selected from the group consisting of a glucosamine formulation, a calming formulation, an urinary tract (UT) formulation, an omega-3 fatty acid formulation, a multivitamin formulation, a probiotic formulation, a weight control formulation, a senior care formulation, and combinations thereof. The foregoing list of animal health promoting products is exemplary only. Other animal health promoting products that are able to be dispersed and/or dissolved into a subject's, e.g., drinking water, and that are substantially colorless, tasteless, and odorless are within the scope of the present invention. Additional animal health promoting products may be designed by combining any of the ingredients disclosed herein or that are otherwise known in the art and processing them according to the methods disclosed herein to form compositions that are substantially colorless, tasteless, and odorless upon contact with a liquid medium.

In the present invention, the glucosamine formulation comprises:

| Ingredient | Amount (mg) |
|---|---|
| Glucosamine | 675-825 |
| Chondroitin Sulfate | 540-660 |
| Methylsulfonylmethane | 540-660 |
| Ascorbic Acid | 108-132 |
| Bromelain | 40.5-49.5 |
| Hyaluronic Acid | 9-11 |
| Manganese | 9-11 |
| L-Glutathione | 1.8-2.2 |
| Dextrose | As required |
| Silicon Dioxide | As required |

In the present invention, the calming formulation comprises:

| | Amount | |
|---|---|---|
| Ingredient | (mg unless otherwise noted) | |
| Colostrum Calming Complex | 13.5-16.5 | |
| Niacinamide | 11.7-14.3 | |
| L-Theanine | 9-11 | |
| Chamomile Flower | 9-11 | |
| Thiamine | 1.35-1.65 | |
| Riboflavin | 1.35-1.65 | |
| Pyridoxine Hydrochloride | 135-165 | mcg |
| Cyanocobalamin | 0.45-0.55 | mcg |

| | |
|---|---|
| Dextrose | As required |
| Silicon Dioxide | As required |

In the present invention, the UT formulation comprises:

| | Amount |
|---|---|
| Ingredient | (mg unless otherwise noted) |
| D-Mannose | 180-220 |
| N-Acetyl-Glucosamine | 180-220 |
| Methionine | 135-165 |
| Bromelain | 90-110 |
| Cranberry Extract | 4.5-5.5 |
| GanedenBC | 90-110 million CFU |
| Dextrose | As required |
| Silicon Dioxide | As required |

In the present invention, the omega-3 fatty acid formulation comprises:

| | Amount | |
|---|---|---|
| Ingredient | (mg unless otherwise noted) | |
| Vitamin A | 900-1100 | IU |
| Vitamin D3 | 90-110 | IU |
| Vitamin E | 1.8-2.2 | IU |
| Thiamine | 1.35-1.65 | |
| Riboflavin | 1.35-1.65 | |
| Niacin | 11.7-14.3 | |
| Pyridoxine Hydrochloride | 135-165 | mcg |
| Vitamin B12 | 0.45-0.55 | mcg |

| | |
|---|---|
| Ascorbic Acid | 108-132 |
| Eicosapentaenoic Acid (EPA) | 81-99 |
| Docosahexaenoic Acid (DHA) | 54-66 |
| Dextrose | As required |
| Silicon Dioxide | As required |

In the present invention, the multi-vitamin formulation comprises:

| | Amount | |
|---|---|---|
| Ingredient | (mg unless otherwise noted) | |
| Calcium (4%) | 90-110 | |
| Phosphorus (3.4%) | 76.5-93.5 | |
| Potassium (0.08%) | 18-22 | |
| Magnesium (0.04%) | 0.9-1.1 | |
| Iron (3200 ppm) | 7.2-8.8 | |
| Copper (20 ppm) | 0.045-0.055 | |
| Manganese (100 ppm) | 0.225-0.275 | |
| Zinc (600 ppm) | 1.35-1.65 | |
| Iodine (20 ppm) | 0.045-0.055 | |
| Vitamin A | 900-1100 | IU |
| Vitamin D3 | 90-110 | IU |
| Vitamin E | 1.8-2.2 | IU |

| | |
|---|---|
| Thiamine | 1.35-1.65 |
| Riboflavin | 1.35-1.65 |
| Niacin | 11.7-14.3 |
| Pyridoxine Hydrochloride | 0.135-0.165 |
| Vitamin B12 | 0.45-0.55 |
| Cobalt | 15.75-19.25 |
| Ascorbic Acid | 108-132 |
| Dextrose | As required |
| Silicon Dioxide | As required |

In the present invention, the probiotic formulation comprises:

| | Amount | |
|---|---|---|
| Ingredient | (mg unless otherwise noted) | |
| Retinol Palmitate | 900-1,100 | IU |
| Cholecaliferol | 90-110 | IU |
| dl-alpha Tocopherol Acetate | 1.8-2.2 | |
| Thiamine | 1.35-1.65 | |
| Riboflavin | 1.35-1.65 | |
| Niacinamide | 11.7-14.3 | |
| Pyridoxine HCl | 135-165 | mch |
| Cyanobalamin | 0.45-0.55 | mcg |

| | |
|---|---|
| Ascorbic Acid | 108-132 |
| Bacillus Coagulans | 90-110 million CFU/gram |
| Dextrose | As required |
| Silicon Dioxide | As required |

In the present invention, the weight control formulation comprises:

| | Amount |
|---|---|
| Ingredient | (mg unless otherwise noted) |
| Phaseolus vulgaris | 450-550 |
| Phase 3 | 450-550 |
| L-Glutamine | 90-110 |
| L-Carnitine | 18-22 |
| L-Lysine | 18-22 |
| Taurine | 18-22 |
| Manganese | 1.8-2.2 |
| Thiamine | 1.35-1.65 |
| Iodine | 0.45-0.65 |
| Biotin | 0.0018-0.0022 |
| Dextrose | As Required |
| Silicon Dioxide | As Required |

In the present invention, the senior care formulation comprises:

| | Amount | |
|---|---|---|
| Ingredient | (mg unless otherwise noted) | |
| L-Lysine | 18-22 | |
| DL-Methionine | 27-33 | |
| Calcium (24%) | 540-660 | |
| Potassium (0.8%) | 18-22 | |
| Manganese (0.08%) | 1.8-2.2 | |
| Iodine (30 ppm) | 67.5-82.5 | mcg |
| Vitamin A | 1350-1650 | IU |
| Vitamin D3 | 90-110 | IU |
| Vitamin E | 9-11 | IU |
| Thiamine | 1.35-1.65 | |
| Riboflavin | 1.35-1.65 | |
| Niacin | 11.7-14.3 | |
| Pyridoxine Hydrochloride | 135-165 | mcg |
| Vitamin B12 | 0.45-0.55 | mcg |

| | |
|---|---|
| Ascorbic Acid | 108-132 |
| L-Glycine | 18-22 |
| L-Carnitine | 18-22 |
| L-Proline | 18-22 |
| Taurine | 18-22 |
| L-Glutamine | 18-22 |
| Dextrose | As required |
| Silicon Dioxide | As required |

In each of the above-identified formulations, the amounts of each ingredient may vary as dictated by a medical professional, such as a veterinarian. Further, the amounts and proportions of each ingredient may further vary as necessary for packaging. Such variations, however, may not be toxic to the subject and must not interfere with the ability of the supplement to be substantially colorless, odorless and tasteless and to disperse and/or dissolve upon contact with the subject's, e.g., drinking water. In a further aspect of this embodiment, the supplement is packaged as a unit dosage form in an amount sufficient to provide the RDA of the supplement to the subject upon consumption of, e.g., the water. In the present invention, a unit dosage form is typically a packet containing the supplement in powder form, which may be provided directly to, e.g., the subject's drinking water. While a powder form is preferred, other forms may be used such as, e.g., stable liquid compositions, water dispersible emulsions, etc., so long as the selected form is dispersible in a liquid medium and is substantially colorless, odorless, and tasteless.

Another embodiment of the present invention is a method for delivering, on a daily basis, a supplement to a dog. The method comprises contacting a source of drinking water for the dog with an amount of the supplement sufficient to provide a recommended daily allowance (RDA) of the supplement to the dog and allowing the dog to consume the supplement-containing water, wherein by consuming the water, the dog ingests the RDA of the supplement.

In one aspect of this embodiment, the supplement is packaged as a unit dosage form in an amount sufficient to provide the RDA of the supplement to the dog upon consumption of the water.

In another aspect of this embodiment, the supplement is a dog health promoting product. Preferably, the dog health promoting product is selected from the group consisting of a glucosamine formulation, a calming formulation, an urinary tract (UT) formulation, an omega-3 fatty acid formulation, a multivitamin formulation, a probiotic formulation, a weight control formulation, a senior care formulation and combinations thereof. Each of the formulations are as disclosed above.

A further embodiment of the present invention is an animal health supplement. The animal health supplement comprises an effective amount of at least one animal health promoting active ingredient, which supplement is sufficient to deliver an effective amount of the active ingredient to the animal via the animal's drinking water, wherein upon contact with the drinking water the supplement disperses within the drinking water to a sufficient degree that the animal is able to ingest the supplement as it consumes the drinking water.

In one aspect of this embodiment, the effective amount is sufficient to deliver a recommended daily amount (RDA) of the active ingredient to the animal upon consumption of the water. As noted above, the RDA is available through various published forms or may be determined by a medical professional on an individual basis for a particular animal or group of animals, based on its/their respective medical needs.

In another aspect of this embodiment, the supplement further comprises one or more acceptable carriers and/or diluents as disclosed herein.

In a further aspect of this embodiment, each ingredient of the supplement is preferably between 7-750 μm in diameter. By providing each or a majority of the ingredients in the supplement with diameters between 7-750 μm, each ingredient is at least dispersible, if not partially or completely soluble, in an aqueous medium as defined above, such as water. And, when such supplements are contacted with, e.g., water, they are dissolved and/or dispersed to such a degree that the subject will not find it objectionable to consume the water due to the color, odor, or taste of the supplements. In this way, the supplements of the invention may be administered to a variety of subjects, large and small, without risk of injury to the caregiver, for example, by virtue of the subject biting the hand/arm of the caregiver as is a common risk associated with administering pills/capsules to, e.g., dogs, and without causing stress to the subject as is often the case when trying to physically force a pill/capsule down the throat of a subject, e.g., a dog.

In an additional aspect of this embodiment, the supplement is packaged in a unit dosage form. The unit dosage form may be packaged as, for example, a once-a-day formulation, a once-a-week formulation, a twice-a-month formulation, or a once-a-month formulation. In this aspect, the unit dosage form may be packaged in any convenient formulation (once-a-day, once-a-week, etc.), so long as when contacted with a liquid medium, e.g., water, the supplement is able to disperse and/or dissolve therein so that the subject will consume the liquid medium without having a negative reaction to its color, odor or taste.

In the present invention, each supplement, when contacted with an aqueous medium, e.g., water, is substantially flavorless, colorless, and odorless. The supplements of the present invention are specifically designed so that the color, flavor and odor of the liquid medium, e.g., water, when contacted with the supplement, are not off-putting to the intended subject, e.g., a dog. While a certain degree of color, odor and/or taste is permissible in the present invention, each characteristic alone, or in combination, may not render the supplement, when contacted with an aqueous medium, unpalatable to the intended subject. Simple observational tests may be used to determine whether a particular supplement meets the requirements of flavorlessness, colorlessness, and odorlessness according to the present invention for a group of subjects.

In a further aspect of this embodiment, the supplement is selected from the group consisting of a glucosamine formulation, a calming formulation, an urinary tract (UT) formulation, an omega-3 fatty acid formulation, a multivitamin formulation, a probiotic formulation, a weight control formulation, a senior care formulation and combinations thereof. Each of these formulations is as disclosed above.

An additional embodiment of the present invention is a dog health supplement. The dog health supplement comprises an effective amount of at least one dog health promoting active ingredient, which supplement is sufficient to deliver a recommended daily allowance (RDA) of the active ingredient to the dog via the dog's drinking water, wherein upon contact with the drinking water the supplement disperses within the drinking water to a sufficient degree that the dog is able to ingest the supplement as it consumes the drinking water.

In one aspect of this embodiment, the supplement comprises one or more acceptable carriers and/or diluents as disclosed herein.

In another aspect of this embodiment, each ingredient of the supplement is preferably between about 7-750 μm in diameter. This characteristic of each ingredient is as disclosed above.

In a further aspect of this embodiment, the supplement is packaged in a unit dosage form in the same manner as disclosed above. Preferably, the unit dosage is a once-a-day formulation, although other unit dosage forms, as disclosed above, may be used.

In an additional aspect of this embodiment, the supplement, when contacted with water, is substantially flavorless, colorless and odorless in the same manner as disclosed above.

In another aspect of this embodiment, the supplement is selected from the group consisting of a glucosamine formulation, a calming formulation, an urinary tract (UT) formulation, an omega-3 fatty acid formulation, a multivitamin formulation, a probiotic formulation, a weight control formulation, a senior care formulation and combinations thereof. Each of these formulations are as disclosed above.

Other embodiments of the present invention include kits for promoting animal, preferably dog, health. These kits comprise the animal, preferably dog, health supplements of the present invention as disclosed previously herein together with instructions for using same.

The kits may also include suitable storage containers, e.g., ampules, vials, tubes, etc., for each supplement and other reagents, e.g., buffers, balanced salt solutions, etc., for use in administering the supplements to subjects. The supplements and other reagents may be present in the kits in any convenient form, such as, e.g., in a stable solution or in a powder form. The kits may further include a packaging container, optionally having one or more partitions for housing the supplements and other optional reagents.

Additional embodiments of the present invention include methods of making any of the animal, preferably dog, health supplements of the present invention as disclosed previously herein. The methods comprise obtaining each ingredient having a diameter between 7-750 μm and combining each ingredient for a sufficient period of time to form a mixture. Exemplary methods for making the supplements of the present invention are provided below in the Examples.

In one aspect, the methods further comprise adding one or more acceptable carriers and/or diluents, as disclosed herein, to the mixture.

In another aspect, the methods further comprise packaging an appropriate amount of the mixture in a container. As one skilled in the art understands, the final mixture may be packaged for bulk transport, in which case the supplements are packaged in any commercially convenient size. Alternatively, the supplements may be packaged in a convenient unit dosage form, e.g., in once-a-day sealed packets. And, the once-a-day sealed packets may be further packaged in any convenient size, e.g., in a box containing 30 packets, for a month long supply.

It will be readily understood that the components of the present invention, as generally described, may be arranged and designed in a wide variety of different configurations or compositions. In addition, a wide variety of fat and/or water-soluble vitamins, minerals, other supplements and combinations thereof, may be used in the methods, kits, and compositions of the invention in varying quantities.

As noted above, different formulations within the scope of the present invention may be fashioned, so long as they are dispersible and/or soluble upon contact with an aqueous medium, e.g., water, and are substantially colorless, odorless, and tasteless in such medium. Thus, exemplary, non-limiting vitamins and minerals that can be used in the methods and supplements of the invention include vitamin B1 (as thiamin or thiamine mononitrate), vitamin B2 (as riboflavin), vitamin B3 (as niacin), vitamin B6 (as pyridoxine or pyridoxine hydrochloride), vitamin B9 (folic acid or folate) or of reduced folate, including but not limited to I-methylfolate, I-5-methyltetrahydrofolate, I-5-methyl-tetrahydrofolic acid, (6S)-tetrahydrofolic acid, (6S)-5-methyl-tetrahydrofolic acid, 5-methyl-(6S)-tetrahydrofolic acid, 5-formyl-(6S)-tetrahydrofolic acid, 10-formyl-(6R)-tetrahydrofolic acid, 5,10-methylene-(6R)-tetrahydrofolic acid, 5,10-methenyl-(6R)-tetrahydrofolic acid, 5-formimino-(6S)-tetrahydrofolic acid, and polyglutamyl derivatives of tetrahydrofolate acid, vitamin B12 (cyanocobalamin, cobalamin, and reduced forms of cobalamin), biotin, vitamin C (ascorbic acid), folacin, niacinamide, calcium, iron, phosphorus, pantothenic acid, iodine, magnesium, magnesium proteinate, zinc, selenium, copper, manganese, chromium, molybdenum, choline, fluoride, chloride, potassium, sodium, biotin and various mixtures or other combinations thereof. The foregoing may be present in the formulations of the present invention in any convenient form, such as elemental forms, salts forms, etc. The foregoing may be obtained from the following representative sources: iron sulfate, zinc sulfate, manganese sulfate, copper sulfate, calcium sulfate, sodium sulfate, copper oxide, magnesium oxide, calcium fluoride, potassium chloride, potassium iodide, sodium chloride, calcium iodate, calcium, magnesium, potassium, sodium or iron phosphate, cobalt carbonate, sodium selenate or silica and salts thereof. A person of ordinary skill in the art would know that alternative sources of such vitamins, minerals or other supplements may be used.

In the present invention, the active ingredients, e.g., the vitamins and minerals used in one or more formulations disclosed herein are, preferably, water soluble. As used herein, water soluble vitamins include the corresponding provitamins and vitamin derivatives such as esters with an action resembling that of the same water-soluble vitamin. Such water-soluble vitamins and/or minerals are optionally present in a solid-state form, such as an amorphous powder or a milled material, for example, a finely milled crystalline material, but can also be present in solution as part of an emulsion of an aqueous solution and edible oils. The active ingredients of the present invention, such as the water-soluble vitamins, preferably have a purity of at least veterinary grade, such as a purity of greater than 90%, even more preferably greater than 95%, particularly preferably greater than 98%, and in formulated form, for example as granules, beadlets or as spray-dried powder.

In the present invention, the supplements may also include fat soluble vitamins, minerals, or other agents. The fat soluble vitamins and minerals that may be employed in the methods and supplements of the invention include, but are not limited to, Vitamin A, Vitamin D, Vitamin E, Vitamin K and tocopherol, the corresponding provitamins and vitamin derivatives such as esters with an action resembling that of vitamin A, D, E or K. For example, vitamin E includes, but is not limited to, natural or synthetic α-, β-, γ- or δ-tocopherol, preferably for natural or synthetic α-tocopherol and for tocotrienol. Vitamin E derivatives are, for example, tocopheryl C1-C20-alkanoic esters such as tocopheryl acetate or tocopheryl palmitate. A person of ordinary skill in the art would know that alternative sources of such vitamins may be used. The fat soluble vitamins and/or minerals are optionally present in a solid-state form, such as an amorphous powder or a milled material, for example, a finely milled crystalline material, but can also be present in solution as part of an emulsion of an aqueous solution and edible oils.

Further, the supplements of the present invention may contain other ingredients, as disclosed above, including but not limited to glucosamine, chondroitin, omega 3 fatty acids, dextrose, mannose, silicon dioxide and colostrum calming complex. Glucosamine and chondroitin may be beneficial for an animal's joints. Omega-3 fatty acids are a family of polyunsaturated fatty acids, some of which are beneficial for the body. Representative examples of nutritionally important omega-3 fatty acids within the scope of the present invention include a-linolenic acid (ALA), eicosapentaenoic acid (EPA) and docosahexaenoic acid (DHA). For pets, omega-3 fatty acids play an important role in many bodily functions, including to control inflammation and maintain joint health and to improve the animal's skin and coat. Additives are optionally present in a solid-state form, such as an amorphous powder or a milled material, for example, a finely milled crystalline material, but can also be present in solution as part of an emulsion of an aqueous solution and edible oils.

Formulations of the invention may be rendered into any pharmaceutically-acceptable form of the vitamins and/or minerals described above, including their salts, which are known by those of skill in the art. For example, useful pharmaceutically-acceptable calcium compounds include any of the well-known calcium supplements, such as calcium carbonate, calcium sulfate, calcium oxide, calcium hydroxide, calcium apatite, calcium citrate-malate, bone meal, oyster shell, calcium gluconate, calcium lactate, calcium phosphate, calcium levulinate, and the like. Pharmaceutically-acceptable magnesium compounds include magnesium stearate, magnesium carbonate, magnesium oxide, magnesium hydroxide and magnesium sulfate. Pharmaceutically-acceptable iron compounds include any of the well-known iron II (ferrous) or iron III (ferric) supplements, such as ferrous sulfate, ferric chloride; ferrous gluconate, ferrous lactate, ferrous tartrate, iron-sugar-carboxylate complexes, ferrous fumarate, ferrous succinate, ferrous glutamate, ferrous citrate, ferrous pyrophosphate, ferrous cholinisocitrate, ferrous carbonate, and the like. The vitamins and/or minerals that may be included in formulations of the invention may be microencapsulated in a coating of fat, microcrystalline cellulose or similar material in order to prevent their degradation under various conditions.

It is to be understood that the formulations of the present invention comprise water-soluble and non-water-soluble ingredients. The former experience an expedited dissolving process due to their small size, while the latter are finely ground so that they may be suspended/dispensed in solution in such a manner that they are not off putting to a subject.

The phrase “water-soluble” as used herein means that the ingredient and/or supplement as a whole dissolves partially or fully (and preferably fully) in an aqueous medium, preferably water.

The terms “suspension” and “dispersion” and related like terms are used interchangeably herein and mean an incorporation of one or more solid-state components into a semi-liquid or liquid medium, and includes colloidal and other types of suspensions. Preferably, the suspension/dispersion is uniform i.e., the components are uniformly present in a semi-liquid or liquid medium.

Because the supplements of the present invention are intended to augment a subject's diet, a person of ordinary skill in the art would readily recognize that the amount of any ingredient that comprises the supplements of the present invention may vary greatly from a very small amount for animals getting a well-balanced diet to an amount near the level where such supplement may have adverse or toxic effects in the animal. A person of ordinary skill in the art can readily determine the upper limit of the amount of each ingredient based upon well-known and published data on the recommended levels, i.e., RDA, of each ingredient as well as the maximum recommended levels of each ingredient.

The supplements may contain one or more active ingredients in percentages that add up to 100%. The supplements may also contain a small amount of residual water. The amount of residual water depends on the drying technology used, which will be evident to a skilled practitioner. Alternatively, other ingredients standard to a vitamin powder composition may be added. For example, vitamin protectors such as sucrose or maltodextrin alone or in combination, and/or antioxidants may be added. The amount of each active ingredient and additive may then be adjusted accordingly.

In addition to the optional additive(s), one or more other compounds, drugs, ingredients and/or materials may be added to the supplements. Regardless of the ingredients selected for each supplement, the ingredients, either individually or in combination, are formulated into pharmaceutically-acceptable dosage forms by conventional methods known to those of skill in the art. See, e.g., Remington, The Science and Practice of Pharmacy (21^{st} Edition, Lippincott Williams and Wilkins, Philadelphia, Pa.).

As set forth above, the supplements of the invention comprise one or more active ingredients optionally in admixture with one or more pharmaceutically-acceptable carriers or diluents. Pharmaceutically acceptable carriers are well known in the art (see, e.g., Remington, The Science and Practice of Pharmacy (21^{st} Edition, Lippincott Williams and Wilkins, Philadelphia, Pa.) and The National Formulary (American Pharmaceutical Association, Washington, D.C.)) and include sugars (e.g., lactose, sucrose, mannitol, and sorbitol), starches, cellulose preparations, calcium phosphates (e.g., dicalcium phosphate, tricalcium phosphate and calcium hydrogen phosphate), sodium citrate, water, aqueous solutions (e.g., saline, sodium chloride injection, Ringer's injection, dextrose injection, dextrose and sodium chloride injection, lactated Ringer's injection), alcohols (e.g., ethyl alcohol, propyl alcohol, and benzyl alcohol), polyols (e.g., glycerol, propylene glycol, and polyethylene glycol), organic esters (e.g., ethyl oleate and tryglycerides), biodegradable polymers (e.g., polylactide-polyglycolide, poly(orthoesters), and poly(anhydrides)), elastomeric matrices, liposomes, microspheres, oils (e.g., corn, germ, olive, castor, sesame, cottonseed, and groundnut), cocoa butter, waxes (e.g., suppository waxes), paraffins, silicones, talc, silicylate, etc. Each pharmaceutically acceptable carrier used in a supplement of the invention must be “acceptable” in the sense of being compatible with the other ingredients of the formulation and not injurious to the subject. Carriers suitable for a selected dosage form are well known in the art, and acceptable carriers for a chosen dosage form can be determined using ordinary skill in the art.

Examples of other additional ingredients and/or materials that may be added to the supplements of the invention include those commonly used in pharmaceutical, e.g., veterinary compositions. These ingredients and materials are well known in the art and include (1) fillers or extenders, such as starches, lactose, sucrose, glucose, mannitol, and silicic acid; (2) binders, such as carboxymethylcellulose, alginates, gelatin, polyvinyl pyrrolidone, hydroxypropylmethyl cellulose, sucrose and acacia; (3) humectants, such as glycerol; (4) disintegrating agents, such as agar-agar, calcium carbonate, potato or tapioca starch, alginic acid, certain silicates, sodium starch glycolate, cross-linked sodium carboxymethyl cellulose and sodium carbonate; (5) solution retarding agents, such as paraffin; (6) absorption accelerators, such as quaternary ammonium compounds; (7) wetting agents, such as cetyl alcohol and glycerol monostearate; (8) absorbents, such as kaolin and bentonite clay; (9) lubricants, such as talc, calcium stearate, magnesium stearate, solid polyethylene glycols, and sodium lauryl sulfate; (10) suspending agents, such as ethoxylated isostearyl alcohols, polyoxyethylene sorbitol and sorbitan esters, microcrystalline cellulose, aluminum metahydroxide, bentonite, agar-agar and tragacanth; (11) buffering agents; (12) excipients, such as lactose, milk sugars, polyethylene glycols, animal and vegetable fats, oils, waxes, paraffins, cocoa butter, starches, tragacanth, cellulose derivatives, polyethylene glycol, silicones, bentonites, silicic acid, talc, salicylate, zinc oxide, aluminum hydroxide, calcium silicates, and polyamide powder; (13) inert diluents, such as water or other solvents; (14) preservatives; (15) surface-active agents; (16) dispersing agents; (17) control-release or absorption-delaying agents, such as hydroxypropylmethyl cellulose, other polymer matrices, biodegradable polymers, liposomes, microspheres, aluminum monostearate, gelatin, and waxes; (18) opacifying agents; (19) adjuvants; (20) wetting agents; (21) emulsifying and suspending agents; (22), solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan; (23) propellants, such as chlorofluorohydrocarbons and volatile unsubstituted hydrocarbons, such as butane and propane; (24) antioxidants; (25) agents which render the formulation isotonic with the blood of the intended recipient, such as sugars and sodium chloride; (26) thickening agents; (27) coating materials, such as lecithin; and (28) sweetening, flavoring, coloring, perfuming and preservative agents. Each such ingredient or material must be “acceptable” in the sense of being compatible with the other ingredients of the formulation and not injurious to the subject and that disperse and/or dissolve upon contact with a liquid medium and that are substantially colorless, odorless, and tasteless.

Formulations of the present invention may be in the form of powders, granules, a solution or a suspension in an aqueous or non-aqueous liquid, an oil-in-water or water-in-oil liquid emulsion, an elixir or syrup, a pastille, a bolus, an electuary or a paste, so long as the form will disperse and/or dissolve upon contact with an aqueous medium and that is substantially colorless, odorless, and tasteless. These formulations may be prepared by methods known in the art, e.g., by means of conventional, mixing, granulation or lyophilization processes.

Solid dosage forms for oral administration (powders, granules, microcapsules, and the like) may be prepared, e.g., by mixing the active ingredient(s) with one or more pharmaceutically-acceptable carriers and, optionally, one or more fillers, extenders, binders, humectants, disintegrating agents, solution retarding agents, absorption accelerators, wetting agents, absorbents, lubricants, and/or coloring agents. They may also be formulated so as to provide slow or controlled release of the active ingredient therein. They may be sterilized by, for example, filtration through a bacteria-retaining filter. These ingredients may also optionally contain opacifying agents and may be of a composition such that they release the active ingredient only, or preferentially, in a certain portion of the gastrointestinal tract, optionally, in a delayed manner.

Liquid dosage forms for oral administration include pharmaceutically-acceptable emulsions, microemulsions, solutions, suspensions, syrups and elixirs. The liquid dosage forms may contain suitable inert diluents commonly used in the art. Besides inert diluents, the oral compositions may also include adjuvants, such as wetting agents, emulsifying and suspending agents, sweetening, flavoring, coloring, perfuming and preservative agents. Suspensions may contain suspending agents.

### EXAMPLES

The following examples will more fully illustrate the embodiments of this invention. It will be understood that the following examples are illustrative and not meant to limit the invention in any way. Those of skill in the art will readily understand that variations of certain of the conditions and/or steps employed in the procedures described in the examples can be used to prepare the supplements. It is also to be understood that more than one formulation may be administered to a non-human subject at the same time, though an appropriate amount of water, as per the instructions, will need to be used to avoid creating a super saturate. All parts, percentages and proportions referred to herein and in the appended claims are by mass unless otherwise illustrated.

### EXAMPLE 1

### Glucosamine Formulation for a Dog

The following ingredients were obtained from commercially available sources and comprise one packet of a composition of the present invention to be administered with 12 oz. of water.

| Ingredient | Amount (mg) |
|---|---|
| Glucosamine | 750 |
| Chondroitin Sulfate | 600 |
| Methylsulfonylmethane | 600 |
| Ascorbic Acid | 120 |
| Bromelain | 45 |
| Hyaluronic Acid | 10 |
| Manganese | 10 |
| L-Glutathione | 2.0 |
| Dextrose | As required |
| Silicon Dioxide | As required |

Each ingredient was passed through one or more sieves of progressively smaller mesh size (about 25-200 mesh) until an average particle diameter of between about 7-750 μm was achieved. The ingredients were then combined in a mixing vessel and mixed. The resulting glucosamine formulation appeared as a fine white to slightly off-white power. The mixture was more crystalline in nature than the other formulations described herein, similar to very finely ground salt. Following mixing, the mixture was then packaged in gross for commercial shipment. Alternatively, the composition is segregated into individual dosage amounts depending on the weight of the dog and its stage of life. A person of ordinary skill in the art can determine the amount of the powdered composition to give to the intended dog based upon knowledge in this area as well as well-known charts provided by several organizations as disclosed herein.

For example, for a dog weighing up to 10 lbs., 0.5 packets of the composition of this example provides many of the RDA of each active ingredient for a dog. Likewise, for a dog weighing 11-50 lbs., 1.0 packet is appropriate, while for a dog weighing more than 50 lbs., 2.0 packets are appropriate.

### EXAMPLE 2

### Calming Formulation for a Dog

The following ingredients were obtained from commercially available sources and comprise one packet of a composition of the present invention to be administered with 12 oz. of water.

| | Amount | |
|---|---|---|
| Ingredient | (mg unless otherwise noted) | |
| Colostrum Calming Complex | 15 | |
| Niacinamide | 13 | |
| L-Theanine | 10 | |
| Chamomile Flower | 10 | |
| Thiamine | 1.5 | |
| Riboflavin | 1.5 | |
| Pyridoxine Hydrochloride | 150 | mcg |
| Cyanocobalamin | 0.5 | mcg |

| | |
|---|---|
| Dextrose | As required |
| Silicon Dioxide | As required |

Each ingredient was passed through one or more sieves of progressively smaller mesh size (about 25-200 mesh) until an average particle diameter of between about 7-750 μm was achieved. The ingredients were then combined in a mixing vessel and mixed. The resulting calming formulation appeared as an off-white fine sand or salt-like powder. Small flecks of light yellow/beige particles that were slightly larger than particles of the powder of Example 1 were also present, as well as some extremely small, dark particles. Following mixing, the mixture was then packaged in gross for commercial shipment. Alternatively, the composition is segregated into individual dosage amounts depending on the weight of the dog and its stage of life. A person of ordinary skill in the art can determine the amount of the powdered composition to give to the intended dog based upon knowledge in this area as well as well-known charts provided by several organizations as disclosed herein.

For a dog weighing up to 10 lbs., 0.5 packets of the composition of this example provides many of the RDA of each active ingredient for a dog. Likewise, for a dog weighing 11-50 lbs., 1.0 packet is appropriate, while for a dog weighing more than 50 lbs., 2.0 packets are appropriate.

### EXAMPLE 3

### Urinary Tract Formulation for a Dog

The following ingredients were obtained from commercially available sources and comprise one packet of a composition of the present invention to be administered with 12 oz. of water.

| | Amount |
|---|---|
| Ingredient | (mg unless otherwise noted) |
| D-Mannose | 200 |
| N-Acetyl-Glucosamine | 200 |
| Methionine | 150 |
| Bromelain | 100 |
| Cranberry Extract | 5.0 |
| GanedenBC (Bacillus coagulans) | 100 million CFU |
| Dextrose | As required |
| Silicon Dioxide | As required |

Each ingredient was passed through one or more sieves of progressively smaller mesh size (about 25-200 mesh) until an average particle diameter of between about 7-750 μm was achieved. The ingredients were then combined in a mixing vessel and mixed. The resulting UT formulation appeared as a fine white powder with some slightly larger granules, similar to a mixture of talc and finely ground white sand. After mixing, the mixture was then packaged in gross for commercial shipment. Alternatively, the composition is segregated into individual dosage amounts depending on the weight of the dog and its stage of life. A person of ordinary skill in the art can determine the amount of the powdered composition to give to the intended dog based upon knowledge in this area as well as well-known charts provided by several organizations as disclosed herein.

For a dog weighing up to 10 lbs., 0.5 packets of the composition of this example provides many of the RDA of each active ingredient for a dog. Likewise, for a dog weighing 11-50 lbs., 1.0 packet is appropriate, while for a dog weighing more than 50 lbs., 2.0 packets are appropriate.

### EXAMPLE 4

### Omega-3 Fatty Acid Formulation for a Dog

The following ingredients were obtained from commercially available sources and comprise one packet of a composition of the present invention to be administered with 12 oz. of water.

| | Amount | |
|---|---|---|
| Ingredient | (mg unless otherwise noted) | |
| Vitamin A | 1000 | IU |
| Vitamin D3 | 100 | IU |
| Vitamin E | 2.0 | IU |
| Thiamine | 1.5 | |
| Riboflavin | 1.5 | |
| Niacin | 13 | |
| Pyridoxine Hydrochloride | 150 | mcg |
| Vitamin B12 | 0.5 | mcg |

| | |
|---|---|
| Ascorbic Acid | 120 |
| Eicosapentaenoic Acid (EPA) | 90 |
| Docosahexaenoic Acid (DHA) | 60 |
| Dextrose | As required |
| Silicon Dioxide | As required |

Each ingredient was passed through one or more sieves of progressively smaller mesh size (about 25-200 mesh) until an average particle diameter of between about 7-750 μm was achieved. The ingredients were then combined in a mixing vessel and mixed. The resulting omega-3 fatty acid formulation appeared as a very fine light yellow to slightly orange colored powder, with a consistency similar to talc. After the mixing, the mixture was then packaged in gross for commercial shipment. Alternatively, the composition is segregated into individual dosage amounts depending on the weight of the non-human subject and its stage of life. A person of ordinary skill in the art can determine the amount of the powdered composition to give to the intended dog based upon knowledge in this area as well as well-known charts provided by several organizations as disclosed herein.

For a dog weighing up to 10 lbs., 0.5 packets of the composition of this example provides many of the RDA of each active ingredient for a dog. Likewise, for a dog weighing 11-50 lbs., 1.0 packet is appropriate, while for a dog weighing more than 50 lbs., 2.0 packets are appropriate.

### EXAMPLE 5

### Multi-Vitamin Formulation for a Dog

The following ingredients were obtained from commercially available sources and comprise one packet of a composition of the present invention to be administered with 12 oz. of water.

| | Amount | |
|---|---|---|
| Ingredient | (mg unless otherwise noted) | |
| Calcium (4%) | 100 | |
| Phosphorus (3.4%) | 85 | |
| Potassium (0.08%) | 20 | |
| Magnesium (0.04%) | 1.0 | |
| Iron (3200 ppm) | 8.0 | |
| Copper (20 ppm) | 0.05 | |
| Manganese (100 ppm) | 0.25 | |
| Zinc (600 ppm) | 1.5 | |
| Iodine (20 ppm) | 0.05 | |
| Vitamin A | 1000 | IU |
| Vitamin D3 | 100 | IU |
| Vitamin E | 2.0 | IU |

| | |
|---|---|
| Thiamine | 1.5 |
| Riboflavin | 1.5 |
| Niacin | 13 |
| Pyridoxine Hydrochloride | 0.15 |
| Vitamin B12 | 0.5 |
| Cobalt | 17.5 |
| Ascorbic Acid | 120 |
| Dextrose | As required |
| Silicon Dioxide | As required |

Each ingredient was passed through one or more sieves of progressively smaller mesh size (about 25-200 mesh) until an average particle diameter of between about 7-750 μm was achieved. The ingredients were then combined in a mixing vessel and mixed. The resulting multi-vitamin formulation appeared as a fine white powder with a salt- or sand-like consistency peppered with dark grey particles. After mixing, the mixture was then packaged in gross for commercial shipment. Alternatively, the composition is segregated into individual dosage amounts depending on the weight of the non-human subject and its stage of life. A person of ordinary skill in the art can determine the amount of the powdered composition to give to the intended dog based upon knowledge in this area as well as well-known charts provided by several organizations as disclosed herein.

For a dog weighing up to 10 lbs., 0.5 packets of the composition of this example provides many of the RDA of each active ingredient for a dog. Likewise, for a dog weighing 11-50 lbs., 1.0 packet is appropriate, while for a dog weighing more than 50 lbs., 2.0 packets are appropriate.

### EXAMPLE 6

### Probiotic Formulation for a Dog

The following ingredients were obtained from commercially available sources and comprise one packet of a composition of the present invention.

| | Amount | |
|---|---|---|
| Ingredient | (mg unless otherwise noted) | |
| Retinol Palmitate | 1000 | IU |
| Cholecaliferol | 100 | IU |
| dl-alpha Tocopherol Acetate | 2.0 | IU |
| Thiamine | 1.5 | |
| Riboflavin | 1.5 | |
| Niacinamide | 13 | |
| Pyridoxine HCl | 150 | mcg |
| Cyanobalamin | 0.5 | mcg |

| | |
|---|---|
| Ascorbic Acid | 120 |
| Bacillus Coagulans | 100 million CFU/gram |
| Dextrose | As required |
| Silicon Dioxide | As required |

Each ingredient was passed through one or more sieves of progressively smaller mesh size (about 25-200 mesh) until an average particle diameter of between about 7-750 μm was achieved. The ingredients were then combined in a mixing vessel and mixed. The resulting probiotic formulation appeared to be similar in texture to crushed chalk or talc and was virtually odorless and flavorless with an off-white or white color. After mixing, the mixture was then packaged in gross for commercial shipment. Alternatively, the composition is segregated into individual dosage amounts depending on the weight of the non-human subject and its stage of life. A person of ordinary skill in the art can determine the amount of the powdered composition to give to the intended dog based upon knowledge in this area as well as well-known charts provided by several organizations as disclosed herein.

For a dog weighing up to 10 lbs., 0.5 packets of the composition of this example provides many of the RDA of each active ingredient for a dog. Likewise, for a dog weighing 11-50 lbs., 1.0 packet is appropriate, while for a dog weighing more than 50 lbs., 2.0 packets are appropriate.

### EXAMPLE 7

### Weight Control Formulation for a Dog

The following ingredients were obtained from commercially available sources and comprise one packet of a composition of the present invention.

| | Amount |
|---|---|
| Ingredient | (mg unless otherwise noted) |
| Phaseolus vulgaris (Phase 2 pet ® | 500 |
| brand) | |
| Phase 3 ™ | 500 |
| L-Glutamine | 100 |
| L-Carnitine | 20 |
| L-Lysine | 20 |
| Taurine | 20 |
| Manganese | 2.0 |
| Thiamine | 1.5 |
| Iodine | 0.5 |
| Biotin | 0.002 |
| Dextrose | As Required |
| Silicon Dioxide | As Required |

Each ingredient was passed through one or more sieves of progressively smaller mesh size (about 25-200 mesh) until an average particle diameter of between about 7-750 μm was achieved. The ingredients were then combined in a mixing vessel and mixed. The resulting weight control formulation appeared to be similar in texture to crushed chalk or talc and was virtually odorless and flavorless with an off-white or white color. The mixture was then packaged in gross for commercial shipment. Alternatively, the composition is segregated into individual dosage amounts depending on the weight of the non-human subject and its stage of life. A person of ordinary skill in the art can determine the amount of the powdered composition to give to the intended dog based upon knowledge in this area as well as well-known charts provided by several organizations as disclosed herein.

For a dog weighing up to 10 lbs., 0.5 packets of the composition of this example provides many of the RDA of each active ingredient for a dog. Likewise, for a dog weighing 11-50 lbs., 1.0 packet is appropriate, while for a dog weighing more than 50 lbs., 2.0 packets are appropriate.

### EXAMPLE 8

### Senior Care Formulation for a Dog

The following ingredients were obtained from commercially available sources and comprise one packet of a composition of the present invention to be administered with 12 oz. of water.

| | Amount | |
|---|---|---|
| Ingredient | (mg unless otherwise noted) | |
| L-Lysine | 20 | |
| DL-Methionine | 30 | |
| Calcium (24%) | 600 | |
| Potassium (0.8%) | 20 | |
| Manganese (0.08%) | 2.0 | |
| Iodine (30 ppm) | 75 | mcg |
| Vitamin A | 1500 | IU |
| Vitamin D3 | 100 | IU |
| Vitamin E | 10 | IU |
| Thiamine | 1.5 | |
| Riboflavin | 1.5 | |
| Niacin | 13 | |
| Pyridoxine Hydrochloride | 150 | mcg |
| Vitamin B12 | 0.5 | mcg |

| | |
|---|---|
| Ascorbic Acid | 120 |
| L-Glycine | 20 |
| L-Carnitine | 20 |
| L-Proline | 20 |
| Taurine | 20 |
| L-Glutamine | 20 |
| Dextrose | As required |
| Silicon Dioxide | As required |

Each ingredient was passed through one or more sieves of progressively smaller mesh size (about 25-200 mesh) until an average particle diameter of between about 7-750 μm was achieved. The ingredients were then combined in a mixing vessel and mixed. The resulting senior care formulation appeared as a very fine white powder, similar to talc or powdered sugar. The mixture was then packaged in gross for commercial shipment. Alternatively, the composition is segregated into individual dosage amounts depending on the weight of the non-human subject and its stage of life. A person of ordinary skill in the art can determine the amount of the powdered composition to give to the intended dog based upon knowledge in this area as well as well-known charts provided by several organizations as disclosed herein.

For a dog weighing up to 10 lbs., 0.5 packets of the composition of this example provides many of the RDA of each active ingredient for a dog. Likewise, for a dog weighing 11-50 lbs., 1.0 packet is appropriate, while for a dog weighing more than 50 lbs., 2.0 packets are appropriate.

### EXAMPLE 9

### Administration of Inventive Formulations

Forty five apparently healthy dogs, each weighing between 20-30 lbs., are sorted into 9 different cohorts, one cohort corresponding to each formulation according to Examples 1-8 above and one control cohort. All dogs are maintained in an AALAC (Association for Assessment and Accreditation of Laboratory Animal Care) accredited facility for the duration of the study. Members of the same cohort are provided with the same once-a-day formulation according to the protocol below:

In the morning, one packet containing a once-a-day formulation according to Examples 1-8 is added to twelve (12) ounces of water in a drinking bowl for each dog in the experimental cohorts. Each formulation, upon contact with the water, immediately disperses. Upon inspection, each water-containing formulation is substantially colorless, odorless, and tasteless. For the control cohort, water only is provided. Each dog has ad libitum access to the water bowl. And, each dog is fed twice per day with 8727C Teklad Certified dog chow.

Throughout the day, the dog is observed drinking water from the bowl until the supplement-containing water is completely consumed by the dog. Thereafter, the water bowl is refilled with water only for the rest of the day. This procedure is repeated daily for three (3) months. At the end of the study, the dogs are examined for over-all health and blood samples are drawn.

Over the course of the 3-month study, all of the dog's behavior toward the supplement-containing water appears to be unchanged compared to water-only. At the end of the study, all dogs appear healthy and have maintained weight and apparent energy levels comparable to the control cohort. Upon analysis, each cohort is expected to have elevated levels of active ingredients compared to control. The respective levels of active ingredients for each experimental cohort is expected to approximate an RDA dose. Moreover, no toxicity is expected to be observed in any of the experimental cohorts compared to control. Thus, the formulations of the present invention are safe and effective for small mammals.

### DOCUMENTS

FREEMAN, L. M. (2006). Disease prevalence among dogs and cats in the United States and Australia and proportions of dogs and cats that receive therapeutic diets or dietary supplements. Journal of the American Veterinary Medical Association 229: 531-534.

All documents cited in this application are hereby incorporated by reference as if recited in full herein.

Although illustrative embodiments of the present invention have been described herein, it should be understood that the invention is not limited to those described, and that various other changes or modifications may be made by one skilled in the art without departing from the scope or spirit of the invention.

1-69. (canceled)

70. A method for delivering a dietary supplement to an animal comprising:
combining a powder of the dietary supplement with an aqueous medium to form a mixture, the powder being dispersed or dissolved within the aqueous medium such that the animal is able to ingest the dietary supplement as the animal consumes the mixture; and
providing, at predetermined time intervals, the mixture for consumption by the animal.

71. The method for delivery the dietary supplement to the animal of claim 70, wherein the animal is selected from the group consisting of dogs, cats, rabbits, guinea pigs, hamsters, ferrets, and horses.

72. The method for delivering the dietary supplement to the animal of claim 70, wherein the predetermined time interval is once daily, once weekly, twice monthly, or once monthly.

73. The method for delivering the dietary supplement to the animal of claim 70, wherein the aqueous medium is a liquid medium selected from a group consisting of water, a balanced salt solution, and an electrolyte solution.

74. The method for delivering the dietary supplement to the animal of claim 73, wherein the mixture is substantially colorless.

75. The method for delivering the dietary supplement to the animal of claim 70, wherein the aqueous medium is a semi-liquid medium.

76. The method for delivering the dietary supplement to the animal of claim 70, wherein the powder has an average particle diameter of about 7 μm to about 750 μm.

77. The method for delivering the dietary supplement to the animal of claim 70, wherein color, odor, or taste of the mixture is not unpalatable to the animal.

78. The method for delivering the dietary supplement to the animal of claim 70, wherein the powder of the dietary supplement is in an amount sufficient to provide the animal a daily dosage within ±10% of a recommended daily allowance (RDA) for the dietary supplement when the mixture is consumed by the animal.

79. The method for delivering the dietary supplement to the animal of claim 70, wherein the powder of the dietary supplement is in an amount sufficient to provide the animal a daily dosage substantially corresponding to a recommended daily allowance (RDA) for the dietary supplement when the mixture is consumed by the animal.

80. The method for delivering the dietary supplement to the animal of claim 70, wherein the powder of the dietary supplement rapidly disperses or dissolves upon contact with the aqueous medium.

81. The method for delivering the dietary supplement to the animal of claim 70, wherein the powder of the dietary supplement is uniformly dispersed or dissolved in the mixture.

82. The method for delivering the dietary supplement to the animal of claim 70, wherein the dietary supplement comprises one or more components selected from a group consisting of water-soluble vitamins and minerals, fat-soluble vitamins and minerals, glucosamine, chondroitin, omega 3 fatty acids, colostrums calming complex, and probiotics.

83. A composition for administration to an animal, comprising:
a powder of a dietary supplement configured to be dispersed or dissolved within an aqueous medium to form a mixture such that the animal is able to ingest the dietary supplement as the animal consumes the mixture, and
wherein the powder is configured such that color, odor or taste of the aqueous mixture is not unpalatable to the animal.

84. The composition of claim 83, wherein the animal is selected from the group consisting of dogs, cats, rabbits, guinea pigs, hamsters, ferrets, and horses.

85. The composition of claim 83, wherein the powder has an average particle diameter of about 7 μm to about 750 μm.

86. The composition of claim 83, wherein the powder is configured to rapidly disperse or dissolve upon contact with the aqueous medium.

87. The composition of claim 83, wherein the dietary supplement comprises one or more components selected from a group consisting of water-soluble vitamins and minerals, fat-soluble vitamins and minerals, glucosamine, chondroitin, omega 3 fatty acids, colostrums calming complex, and probiotics.

88. A kit for administration of a dietary supplement to an animal comprising:
a powder of a dietary supplement configured to be dispersed or dissolved within an aqueous medium containing water to form a mixture, such that the animal is able to ingest the dietary supplement as the animal consumes the mixture, wherein color, odor or taste of the aqueous mixture is not unpalatable to the animal; and
a storage container containing an amount of the powder of the dietary supplement sufficient to provide the animal a daily dosage substantially corresponding to a recommended daily allowance (RDA) for the dietary supplement when the mixture is consumed by the animal.

89. The kit of claim 88, wherein the amount of the powder of the dietary supplement contained in the storage container is sufficient to provide the animal a daily dosage within ±10% of a recommended daily allowance (RDA) for the dietary supplement when the mixture is consumed by the animal.
